(19) United States
(12) Patent Application Publication
(10) Pub. No.: US 2006/0275308 A1
(43) Pub. Date: Dec. 7, 2006

(54) Combination of gallium compounds with nonchemotherapeutic anticancer agents in the treatment of neoplasia

(75) Inventors: Raymond P. Warrell JR., Westfield, NJ (US); Stefan C. Grant, New York, NY (US); Bob D. Brown, Long Hill Township, NJ (US)

Correspondence Address: KENYON & KENYON LLP, ONE BROADWAY, NEW YORK, NY, 10004, US

(73) Assignee: Genta Incorporated

(21) Appl. No.: 11/501,248

(22) Filed: Aug. 7, 2006

Related U.S. Application Data
(63) Continuation of application No. 10/750,742, filed on Dec. 31, 2003, now abandoned.
(60) Provisional application No. 60/437,275, filed on Dec. 31, 2002.

(51) Int. Cl. A61K 39/395; A61K 38/21; A61K 38/20; A61K 38/17; A61K 31/454; A61K 31/203; A61K 33/36; A61K 33/24; A61K 31/553

(52) U.S. Cl. 424/155.1; 424/617; 424/85.2; 424/85.7; 514/44; 514/12; 514/323; 514/559; 424/623

(57) ABSTRACT

The present invention relates to a combination of a pharmaceutical composition comprising a gallium compound, especially gallium nitrate, and one or more nonchemotherapeutic anticancer agents (NCAA) including antibodies, antisense molecules, anti-telomerase agents, aptamers, biologic response modifiers, bisphosphonates, cytotoxic fusion proteins, immunomodulatory agents, immunostimulatory agents, molecular decoys, molecular inhibitors, proteasome inhibitors, protein kinase inhibitors, retinoids, transcription factors and arsenic compounds, for the treatment of neoplasic disease in a mammal in need of treatment thereof.

### FIELD OF THE INVENTION

The present invention relates to a combination of a pharmaceutical composition comprising a gallium compound, especially gallium nitrate, and one or more nonchemotherapeutic anticancer agents (NCAA) selected from the group including but not limited to antibodies, antisense molecules, anti-telomerase agents, aptamers, biologic response modifiers, bisphosphonates, cytotoxic fusion proteins, immunomodulatory agents, immunostimulatory agents, molecular decoys, molecular inhibitors, proteasome inhibitors, protein kinase inhibitors, retinoids, transcription factors or arsenic compounds, for the treatment of neoplasic disease in a mammal in need of treatment thereof.

### BACKGROUND OF THE INVENTION

The pleiotropic effects of gallium include anticancer activity as well as decreased bone resorption and increased calcium accretion in bone. Gallium is the second metal ion to be used in cancer treatment after platinum. Collery, P., Keppler, B., Madoulet, C., and Desoize, B., Gallium in cancer treatment. (Critical Reviews in Oncology/Hematology 42 (2002) 283-296; incorporated herein by reference). Its pleiotropic effects include modification of the tertiary structure of DNA as well as inhibition of DNA synthesis, modulation of protein synthesis, inhibition of various enzyme activities, including ATPases, DNA polymerases, and tyrosine-specific protein phosphatases among others. Gallium alters the permeability of plasma membranes and mitochondrial functions. Gallium compounds available include gallium nitrate, gallium chloride and gallium maltolate, as well as new compounds such as doxorubicin-gallium-transferrin conjugate and tris(8-quinolinolato)Ga(III) which demonstrate unusual properties. In addition to the effects on bone resorption noted above, the anticancer activity of gallium salts has also been demonstrated. Gallium induces tumor fibrosis with extended duration of administration and exhibits a synergistic effects with other anticancer agents. These properties imply a utility for gallium in the treatment cancer, bone metastases in cancer patients, and other neoplasias.

Gallium is a Group IIIa metal of the periodic table with an atomic number of 31, atomic weight of 69.72 and melting point of 29.78° C. Gallium was discovered in 1875 by Lecoq de Boisbaudran, who noted the predicted properties similar to aluminum as predicted by Mendeleyev. In most compounds, the oxidation state of gallium is +3 and gallium exhibits physicochemical behavioral properties similar to Fe⁺⁺⁺ in its electric charge, ion diameter, coordination number and electronic configuration. The radioactive isotopes, Ga⁶⁷ and Ga⁶⁸, are taken up as compounds by metatastases in bone and therefore show potential for the study of bone cancer. The anticancer properties of gallium were first described in 1971 by Hart et al. (Antitumor activity and toxicity of salts of inorganic group 3a metals: aluminum, gallium, indium, and thallium, Proc. Natl. Acad Sci USA 1971; 68:1623-1626; Toxicity and antitumor activity of gallium nitrate and periodically related metal salts, J. Natl. Cancer Inst. 1971; 47:1121-1127.)

Gallium nitrate has been demonstrated to be cytostatic as well as cytotoxic to cells in vitro, with growth inhibition occurring with chronic exposure at low concentrations (10 μg/ml). Exposure of duration longer than 24 hours induces both cellular cytoxicity and inhibition of growth at concentrations in excess of 50 μg/ml. A distinctive feature of the cytotoxic effects of gallium is its effect on cells in both the exponential growth phase and stationary phase, which is unusual for cytotoxic drugs. Cell inhibition by gallium is both dose and time dependent

Warrell, Jr. et al. have shown that gallium salts, especially gallium nitrate, are useful in treatments for regulating calcium resorption from bone in certain bone diseases and hypercalcemia (U.S. Pat. No. 4,529,593, incorporated by reference herein).

### SUMMARY OF THE INVENTION

The present invention provides a method for the treatment of neoplastic disease, comprising the steps of administering a gallium compound preferably gallium nitrate and administering an NCAA preferably an antibody, to a patient in need thereof. The gallium compound and antibody can be administered together or at predetermined intervals. Preferably the NCAA is administered via the generally recommended route and at the generally recommended dose.

In one aspect, the present invention provides a combination of a gallium compound, especially gallium nitrate, and-at least one NCAA selected from the group consisting of an antibody, an antisense molecule, an anti-telomerase agent, an aptamer, a biologic response modifier, a bisphosphonate, a cytotoxic fusion protein, an immunomodulatory agent, an immunostimulatory agent, a molecular decoy, a molecular inhibitor, a proteasome inhibitor, a protein kinase inhibitor, a retinoid, a transcription factor and an arsenic compound; for the treatment of neoplasic disease in a mammal in need of treatment thereof.

In one aspect, the present invention provides a combination of a gallium compound, especially gallium nitrate, and at least one NCAA wherein the NCAA is a small molecule.

In another aspect, the present invention provides a combination of a gallium compound, especially gallium nitrate, and at least one NCAA selected from the group consisting of an antibody, an antisense molecule, an anti-telomerase agent, an aptamer, a biologic response modifier, a bisphosphonate, a cytotoxic fusion protein, an immunomodulatory agent, an immunostimulatory agent, a molecular decoy, a molecular inhibitor, a proteasome inhibitor, a protein kinase inhibitor, a retinoid, a transcription factor or an arsenic compound, wherein the preferred therapeutic dose of gallium nitrate is a daily dose ranging between about 100 mg/m²/d and about 400 mg/m²/d over about 3 days to about 8 days, more preferably between about 250 mg/m²/d and about 350 mg/m²/d over about 5 days to about 7 days, and most preferably about 300 mg/m²/d over about 7 days.

In yet another aspect, the present invention provides a combination of a gallium compound, especially gallium nitrate, and at least one antibody, preferably an antibody selected from the group consisting of a monoclonal antibody, a chimeric antibody, a genetically engineered antibody, a bispecific antibody, an antibody fragment, a single-chain antibody, an scFv fragment, an Fab fragment, an F(ab)′ fragment, an (Fab)′₂ fragment, a chimeric antibody, e.g., a humanized antibody, e.g., alemtuzumab (Campath®), cetuximab (IMC-C225, Erbitux™), epratuzumab (LL2, hLL2, LymphoCide®), gemtuzumab ozogamicin (Mylotarg®), ibritumomab tiuxetan (Zevalin®), rituximab (Rituxan®), tositumomab (Bexxar®), trastazumab (Herceptin®), or anti-CD19/anti-CD3 single-chain bispecific antibody (bscCD19xCD3), wherein the most particularly preferred embodiment of the NCAA of the present invention is rituximab (Rituxan®).

In yet another aspect, the present invention provides a combination of a gallium compound, especially gallium nitrate, and rituxamab. The preferred dose of rituximab is between about 250 mg/m² and about 425 mg/m², more preferably between about 325 mg/m² and about 400 mg/m² and most preferably about 375 mg/m². The preferred frequency of administration is between about once weekly to about once monthly, at a more preferred frequency of between about once weekly to about twice monthly and at a most preferred frequency of about once weekly, for a preferred duration of about 1 to about 12 doses, most preferably for about 4 to about 8 doses.

In still another aspect, the present invention provides a combination of gallium nitrate and alemtuzumab. The preferred dosage of alemtuzumab is between about 3 mg/d and about 30 mg/d, more preferably between about 10 mg/d and about 30 mg/d, and most preferably about 30 mg/d about three times weekly, wherein the maximum preferred dosage of about 30 mg/d about three times weekly from an initial preferred dosage of about 3 mg/d about three times weekly is accomplished preferably between about 3 days to about 14 days, more preferably between about 3 days to about 10 days and most preferably between about 3 days to about 7 days, wherein the most preferred frequency of administration of alemtuzumab is about three times per week on alternate days for a maximum preferred dose of not more than 90 mg/wk for a preferred duration of up to about 12 weeks.

In still another aspect, the present invention provides a combination of gallium nitrate and cetuximab (IMC-C225, Erbitux™). The preferred initial dose of cetuximab is about 250 mg/m² to about 400 mg/m², and a particularly dose is about 400 mg/m² followed by weekly maintenance doses of about 250 mg/m².

In still another aspect, the present invention provides a combination of gallium nitrate and epratuzumab (LL2, hLL2, LymphoCide®). The preferred dose of epratuzumab is between about 320 mg/m² and about 520 mg/m², more preferably between about 340 mg/m² and about 500 mg/m², and most preferably between about 360 mg/m² and about 480 mg/m² by weekly infusion.

In still another aspect, the present invention provides a combination of gallium nitrate and gemtuzumab ozogamicin (Mylotarg®). The preferred dose of gemtuzumab ozogamicin is between about 7 mg/m² and about 11 mg/m², the more preferred dose is between about 8 mg/m² and about 1 mg/m², and the most preferred dose is about 9 mg/m², administered as a 2-hour intravenous infusion, for a total treatment course of about 2 doses given about 14 days apart.

In still another aspect, the present invention provides a combination of gallium nitrate and ibritumomab tiuxetan (Zevalin®) and rituximab. The rituximab is administered at a preferred dose of about 250 mg/m², followed by In¹¹¹-labeled ibritumomab tiuxetan at the preferred dose of about 5.0 mCi (1.6 mg total antibody dose) injected intravenously over a period of 10 minutes, followed by a second administration of rituximab at a preferred dose of about 250 mg/m², followed by Y⁹⁰-labeled ibritumomab tiuxetan administered at a preferred dose of between about 0.3 mCi/kg (11.1 MBq/kg) to about 0.4 mCi/kg (14.8 MBq/kg) actual body weight injected intravenously over a period of 10 minutes.

In still another aspect, the present invention provides a combination of gallium nitrate and tositumomab (Bexxar®). The preferred dose of tositumomab is 450 mg intravenously over 1 hr, followed by 35 mg of tositumomab radiolabeled with 5 mCi of iodine-131 over 0.5 hr.

In still another aspect, the present invention provides a combination of gallium nitrate and trastazumab (Herceptin®). The preferred initial dose of trastazumab is between about 3 mg/kg to about 5 mg/kg, more preferably, between about 3.5 mg/kg to about 4.5 mg/kg, and most preferably about 4 mg/kg, administered as a 90-minute intravenous infusion. The preferred weekly maintenance dose of trastuzumab is between about 1 mg/kg to about 3 mg/kg, more preferably, between about 1.5 mg/kg to about 2.5 mg/kg, and most preferably about 2 mg/kg administered over about a 30-minute period as an intravenous infusion.

In still another aspect, the present invention provides a combination of gallium nitrate and anti-CD19/anti-CD3 single-chain bispecific antibody (bscCD19xCD3).

In yet a further aspect, the present invention provides a combination of a gallium compound, especially gallium nitrate, and a NCAA selected from the group consisting of an antibody, an antisense molecule e.g., G3139 (oblimersen sodium, Genasense™), an anti-telomerase agent, e.g., antisense small molecule or oligomer (e.g., GRN163), an aptamer, a biologic response modifier, e.g., interleukin-2 (IL-2, aldesleukin, Proleukin®), interleukin-11 (IL-11), interleukin-12 (IL-12), or interferon-alpha2a (IFN-α2a), a bisphosphonate, e.g., zoledronic acid (Zometa®), a cytotoxic fusion protein, e.g., denileukin diftitox (Ontak®), an immunomodulatory agent, e.g., thalidomide (Thalomid®), an immunostimulatory agent, e.g., granulocyte-macrophage-colony stimulating factor (GM-CSF, Leukine®), a molecular decoy, a molecular inhibitor, e.g., P-glycoprotein inhibitor (PSC-833), a proteasome inhibitor, e.g., bortezomib (Velcade®), a protein kinase inhibitor, including a protein tyrosine kinase inhibitor, e.g., imatinib mesylate (Gleevec®), or gefitinib (Iressa®), and a protein kinase C inhibitor, e.g., ruboxistaurin mesylate (LY333531®), a retinoid, e.g., bexarotene (Targretin®), or tretinoin (Vesanoid®), a transcription factor, e.g., nuclear factor-kappa B (NF-κB), and an arsenic compound, e.g., arsenic trioxide (Trisenox®), for the treatment of neoplasic disease in a mammal in need of treatment thereof.

The present invention also provides the gallium compounds, NCAA compounds and formulations thereof, adapted for use in the manufacture of drugs for administration to patients having neoplastic disease.

The present invention may be used in combination with chemotherapeutic therapies.

### DETAILED DESCRIPTION OF THE INVENTION

The present invention comprises administering a pharmaceutical composition comprising a gallium compound, preferably gallium nitrate, and at least one NCAA selected from the group consisting of an antibody, an antisense molecule, an anti-telomerase agent, an aptamer, a biologic response modifier, a bisphosphonate, a cytotoxic fusion protein, an immunomodulatory agent, an immunostimulatory agent, a molecular decoy, a molecular inhibitor, a proteasome inhibitor, a protein kinase inhibitor, a retinoid, a transcription factor or an arsenic compound, preferably arsenic trioxide, for the treatment of a neoplasm in a mammal in need of treatment thereof.

Another aspect of the present invention is a pharmaceutical composition comprising a gallium compound, preferably gallium nitrate, for administration to a mammal in need thereof, in combination with at least one NCAA selected from the group consisting of an antibody, an antisense molecule, an anti-telomerase agent, an aptamer, a biologic response modifier, a bisphosphonate, a cytotoxic fusion protein, an immunomodulatory agent, an immunostimulatory agent, a molecular decoy, a molecular inhibitor, a proteasome inhibitor, a protein kinase inhibitor, a retinoid, a transcription factor or an arsenic compound, preferably arsenic trioxide.

As used herein, neoplasm refers to “new growth; an abnormal tissue that grows by cellular proliferation more rapidly than normal and continues to grow after the stimuli that initiated the new growth cease. Neoplasms show partial or complete lack of structural organization and functional coordination with the normal tissue; they usually form a distinct mass of tissue.” Stedman's Medical Dictionary, 27^{rd} Ed., Lippincott, Williams & Wilkins, pub., 2000. As used herein, neoplasm includes malignant neoplastic disease wherein the neoplasm is selected from the group including but not limited to solid tumors, wherein solid tumors include but are not limited to gastric carcinoma, pancreatic carcinoma, head and neck cancer, sarcoma, breast carcinoma, lung carcinoma, prostate carcinoma, colon carcinoma, ovarian carcinoma, central nervous system tumor, neuroblastoma, glioblastoma multiforme or melanoma; hematologic malignancies, wherein the hematologic malignancies include but are not limited to the range of acute and chronic leukemias and lymphomas, e.g., acute lymphoblastic leukemia (ALL), acute myelogenous leukemia (AML), acute promyelocytic leukemia (APL), chronic myelogenous leukemia (CML), chronic lymphocytic leukemia (CLL), cutaneous T-cell lymphoma (CTCL), or non-Hodgkin's lymphoma (NHL); plasma cell disorders including but not limited to myeloma, Waldenström's macroglobulinemia, as well as myelodysplastic disease and myeloproliferative disease.

The compounds of the present invention include pharmacologically acceptable gallium compounds and salts, esters and solvates, thereof. Examples of gallium compounds useful in the practice of the present invention include gallium nitrate, gallium chloride, gallium maltolate, gallium gluconate, gallium palmitate and each of the pharmaceutically acceptable salts, esters and solvates thereof. In one preferred embodiment of the present invention the gallium compound is a hydrate of gallium nitrate, most preferably gallium nitrate monohydrate. The preferred gallium compounds are readily bioavailable and cause limited side-effects, e.g., membrane irritation.

The preferred dose of gallium nitrate is a daily dose from about 100 mg/m²/d to about 400 mg/m²/d for from 3 days to about 8 days, more preferably a dose from about 250 mg/m²/d to about 350 mg/m²/d for from about 5 days to about 7 days, and most preferably about 300 mg/m²/d over about 7 days.

Administration of the gallium compound in the combination as provided in the present invention can be by any means known in the art, including but not limited to enteral administration including oral administration and rectal administration, and parenteral administration, including but not limited to intravenous, intramuscular, subcutaneous, intraperitoneal, or intravenous administration. The compound can be administered as a bolus, or as an infusion. For the most preferred gallium compound of the present invention, gallium nitrate, the preferred means of administration is by continuous intravenous infusion.

As used herein, NCAA useful in the practice of the present invention include molecules selected from the group including but not limited to antibodies, antisense molecules, anti-telomerase agents, aptamers, biologic response modifiers, bisphosphonates, cytotoxic fusion proteins, immunomodulatory agents, immunostimulatory agents, molecular decoys, molecular inhibitors, proteasome inhibitors, protein kinase inhibitors, retinoids, transcription factors and arsenic compounds. The NCAA of the present invention exclude conventional chemotherapeutic agents. It will be understood by one of ordinary skill in the art that the NCAAs of the present invention have specific and generally well defined molecular targets, while conventional chemotherapeutic agents generally are limited in their selectivity of specific cells and/or molecular target sites, such as DNA sites generally. As a term understood by one skilled in the art, chemotherapeutic agents excluded from the present invention include vinca alkaloids, camptothecan, taxane, or platinum analogues, including vincristine, vinblastine, vinorelbine, vindesine, paclitaxel, docetaxel, 5-fluorouracil, cisplatin, carboplatin, iranotecan, topotecan or cyclophosphamide. While the present invention comprises the use of a gallium compound in combination with a NCAA, nothing in this application should be construed as precluding the practice of the present invention together with conventional chemotherapy.

In a preferred embodiment of the present invention the NCAA is an antibody. As used herein, antibody refers to molecules which include a complete antibody, an antibody fragment, including Fab fragment, F(ab)′, (Fab)′₂, single chain antibody, or peptides. A more preferred embodiment of the antibody of the present invention includes mouse antibodies, rat antibodies, rabbit antibodies, or human antibodies, or fragments thereof, e.g., polyclonal or monoclonal antibodies.

In preferred embodiments of the present invention the antibody includes a monoclonal antibody, a chimeric antibody, a genetically engineered antibody, a bispecific antibody, a single-chain antibody, a scFv fragment, an Fab fragment, an F(ab)′ fragment, an (Fab)′₂ fragment, a humanized antibody, or an antibody fragment thereof, including, e.g., alemtuzumab (Campath®), cetuximab (IMC-C225, Erbitux™), edrecolomab (Panorex®), epratuzumab (LL2, hLL2, LymphoCide®), gemtuzumab ozogamicin (Mylotarg®), ibritumomab tiuxetan (Zevalin®), rituximab (Rituxan®), tositumomab (Bexxar®), trastazumab (Herceptin®), or anti-CD19/anti-CD3 single-chain bispecific antibody (bscCD19xCD3). In a more preferred embodiment of the present invention the antibody is rituximab (Rituxan®).

Various procedures are known in the art for the production of such antibodies and fragments. As would be known to the skilled artisan, techniques used for preparation of monoclonal antibodies, include but are not limited to, the hybridoma technique (Kohler & Milstein, Nature, 256:495-497 (1975)), the trioma technique, the human B-cell hybridoma technique (Kozbor et al., Immunology Today 4:72, (1983)), and the EBV-hybridoma technique to produce human monoclonal antibodies (Cole, et al., 1985, in Monoclonal Antibodies and Cancer Therapy, Alan R. Liss, Inc., pp. 77-96). Techniques described for the production of single chain antibodies (U.S. Pat. No. 4,946,778, incorporated herein by reference) are adapted to produce single chain antibodies. Techniques described for the production of phage display libraries are adapted for the production of single chain Fv antibody-fragments (Winter, G. et al., Making antibodies by phage display technology, Annu. Rev. Immunol. 12, 433-455 (1994); Vaughan et al., Human antibodies with sub-nanomolar affinities isolated form a large non-immunised phage display library, Nature Biotechnology, 14, 309-314 (1996)). Also, transgenic mice are used to express humanized antibodies of this invention. In one strategy, the human heavy and light chain immunoglobulin gene complexes are introduced into a mouse germ line to yield animals whose antibody production is purely human. Epitope binding components of the present invention refer to proteins consisting of one or more polypeptides substantially encoded by genes of the immunoglobulin superfamily (e.g., see “The Immunoglobulin Gene Superfamily,” A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference). Included within the scope of this invention are bispecific antibodies that are formed by joining two epitope binding components that have different binding specificities. In preferred embodiments of the invention, the epitope binding component is encoded by immunoglobulin genes that are “chimeric” or “humanized” (see, generally, Queen (1991) Nature 351:501, which is incorporated herein by reference).

As used herein, rituximab (Rituxane), is a monoclonal antibody which is a human-mouse chimeric anti-CD20 monoclonal antibody, i.e., a genetically engineered antibody from portions of mouse and human antibodies for the treatment of patients with relapsed or refractory, low grade or follicular, CD20-positive, B-cell non-Hodgkin's lymphoma (NHL). The antibody is an IgG1 kappa immunoglobulin containing murine light-and heavy-chain variable region sequences and human constant region sequences. Rituximab is composed of two heavy chains of 451 amino acids and two light chains of 213 amino acids (based on cDNA analysis) and has an approximate molecular weight of 145 kD.

In accordance with the present invention, the preferred dosage of rituximab would be known to one of skill in the art as suggested by the Physicians' Desk Reference, 56^{th} Ed. (2002) or a similar reference. The preferred dosage of rituximab is from about 250 mg/m² to about 425 mg/m², more preferably from about 325 mg/m² to about 400 mg/m² and most preferably about 375 mg/m². The preferred frequency of administration is between about once weekly to about once monthly, at a more preferred frequency of between about once weekly to about twice monthly and at a most preferred frequency of about once weekly, for a preferred duration of about 1 to about 12 doses, most preferably for about 4 to about 8 doses. Patients who subsequently develop progressive disease may be retreated with rituximab about 375 mg/m² intravenous infusion preferably at a frequency of about once weekly for about 4 doses. The present invention also encompasses pharmaceutical compositions comprising an effective amount of rituximab to be administered in combination with the gallium compound of the present invention.

In another embodiment, the present invention also encompasses the combination of a gallium compound, preferably gallium nitrate, and at least one other NCAA to be administered with or in addition to rituximab.

As used herein, alemtuzumab (Campath®) refers to a recombinant DNA-derived humanized monoclonal antibody, used as an injectable treatment for B-cell chronic lymphocytic leukemia (B-CLL). Alemtuzumab binds to the CD52 antigen, a “cluster of differentiation” cell-surface protein on normal and malignant B and T Iymphocytes, NK cells, monocytes, macrophages, and tissues of the male reproductive system. Alemtuzumab induces antibody-dependent lysis, or killing, thereby removing malignant lymphocytes from the blood, bone marrow, and other affected organs.

In accordance with the present invention, the preferred dosage of alemtuzumab would be known to one of skill in the art as suggested by the Physicians' Desk Reference, 56^{th} Ed. (2002) or a similar reference. The preferred dosage of alemtuzumab is between about 3 mg/d and about 30 mg/d about three times weekly, more preferably between about 10 mg/d and about 30 mg/d about three times weekly, and most preferably about 30 mg/d about three times weekly. Achievement of the preferred dosage of about 30 mg/d about three times weekly from an initial preferred dosage of about 3 mg/d about three times weekly which is accomplished preferably over about 3 days to about 14 days, more preferably over about 3 days to about 10 days and most preferably over about 3 days to about 7 days. The most preferred frequency of administration of alemtuzumab is about three times per week administered no more often than every other day for a preferred maximum dose of not more than 90 mg/wk for a preferred duration of up to about 12 weeks. The preferred route of administration is intravenously more preferably by intravenous infusion over about a 2 hr period. The present invention also encompasses a pharmaceutical composition comprising an effective amount of alemtuzumab to be administered in combination with a gallium compound in accordance with the methods of the present invention.

As used herein, cetuximab (IMC-C225, Erbitux™) refers to a chimeric monoclonal antibody, part mouse and part human, that binds specifically to epidermal growth factor receptor (EGFr) and blocks the ability of epidermal growth factor (EGF) to initiate receptor activation and signaling to the tumor. Cetuximab targets and inhibits EGFr, which is associated with tumor cell growth in a number of EGFr-positive solid malignant tumors. EGFr is over-expressed in more than 35% of all solid malignant tumors. The blockade inhibits tumor growth by interfering with the effects of EGFr activation including tumor invasion and metastases, cell repair and angiogenesis. The preferred route of administration is parenteral with a preferred initial dosage of about 250 mg/m² to about 400 mg/r², and a particularly preferred dosage of about 400 mg/m² followed by weekly maintenance doses of about 250 mg/m². The present invention also encompasses pharmaceutical compositions comprising an effective amount of cetuximab to be administered with a gallium compound in accordance with the methods of the present invention.

As used herein, gemtuzumab ozogamicin (Mylotarg®) refers to a recombinant humanized monoclonal antibody conjugated with the cytotoxic antitumor antibiotic, calicheamicin. The antibody portion binds specifically to the CD33 antigen found on the surface of leukemic blasts and immature normal cells of myelomonocytic lineage but not on pluripotent hematopoietic stem cells, forming a complex that the target cell internalizes. Inside the cell, the calicheamicin portion of the conjugate is released by hydrolysis. Calicheamicin migrates into the cell nucleus and binds to DNA, producing double-strand breaks that result in cell death. Gemtuzumab ozogamicin preferably is used to treat patients with CD33-positive acute myeloid leukemia in first relapse who are 60 years of age or older and are not considered to be candidates for cytotoxic chemotherapy. The preferred dosage is between about 7 mg/m² and about 11 mg/m², the more preferred dosage is between about 8 mg/m² and about 10 mg/m², and the most preferred dosage is about 9 mg/m², administered as a 2-hour intravenous infusion, for a total treatment course of about 2 doses given about 14 days apart. The present invention also encompasses pharmaceutical compositions comprising an effective amount of gemtuzumab ozogamicin to be administered with a gallium compound in accordance with the methods of the present invention.

As used herein, epratuzumab (LL2, LymphoCide®) refers to a humanized monoclonal antibody that targets CD22 receptor on mature and malignant B lymphocytes, including NHL. The preferred dose is between about 320 mg/m² and about 520 mg/m², the more preferred dose is between about 340 mg/m² and about 500 mg/m², and the most preferred dose is between about 360 mg/m² and about 480 mg/m² by weekly infusion.

As used herein, ibritumomab tiuxetan (Zevalin®) refers to an immunoconjugate resulting from a stable thiourea covalent bond between the monoclonal antibody, ibritumomab, and the linker-chelator, tiuxetan, [N-[2-bis(carboxymethyl)amino]-3-(p-isothiocyanatophenyl)-propyl]-[N-[2-bis(carboxymethyl)amino]-2-(methyl)-ethyl]glycine. This linker-chelator provides a high affinity, conformationally restricted chelation site for radiopharmaceuticals, Indium¹¹¹ or Yttrium⁹⁰. The antibody moiety is ibritumomab, a murine IgG, kappa monoclonal antibody directed against the CD20 antigen, which is found on the surface of normal and malignant B lymphocytes. Ibritumomab tiuxetan binds specifically to the CD20 antigen (human B-lymphocyte-restricted differentiation antigen, Bp35). The CD20 antigen is expressed on pre-B and mature B lymphocytes and on >90% of B-cell non-Hodgkin's lymphomas (NHL). The CD20 antigen is not shed from the cell surface and does not internalize upon antibody binding. Ibritumomab tiuxetan is used in the treatment of relapsed or refractory-low grade, follicular, or transformed B-cell non-Hodgkins lymphoma (NHL) including patients with rituximab-refractory follicular NHL.

The ibritumomab tiuxetan therapeutic regimen preferably is administered in combination with rituximab in two steps: The first step comprises an intravenous infusion of rituximab at a preferred dose of about 250 mg/m² at an initial rate of about 50 mg/hr, with an escalation of the infusion rate in 50 mg/hr increments every 30 minutes, to a preferred maximum of about 400 mg/hr, as tolerated, followed by In¹¹¹-labeled ibritumomab tiuxetan within about 4 hours following completion of the rituximab dose. A preferred dose of about 5.0 mCi (1.6 mg total antibody dose) of In¹¹¹-labeled ibritumomab tiuxetan is injected intravenously over a period of 10 minutes. The second step follows the first by seven to nine days and consists of a second infusion of rituximab at the same preferred dose of about 250 mg/m² administered intravenously at a preferred initial rate of 100 mg/hr, increased by about 100 mg/hr increments at 30 minute intervals, to a preferred maximum of about 400 mg/hr, as tolerated, followed by Y⁹⁰-labeled ibritumomab tiuxetan within about 4 hours following completion of the rituximab dose of step 2. Y⁹⁰-labeled ibritumomab tiuxetan is administered at a preferred dose of between about 0.3 mCi/kg (11.1 MBq/kg) to about 0.4 mCi/kg (14.8 MBq/kg) actual-body weight (depending upon platelet counts of 100,000-149,000 cells/mm³ and >150,000 cells/mm³, respectively), by intravenous injection over a period of about 10 minutes. The administered dose of Y⁹⁰-labeled ibritumomab tiuxetan is most preferred not to exceed a dose of 32.0 mCi (1,184 MBq), regardless of the patient's body weight. The present invention also encompasses pharmaceutical compositions comprising an effective amount of ibritumomab tiuxetan to be administered in combination with a gallium compound in accordance with the methods of the present invention.

As used herein, tositumomab (Bexxar®) refers to an anti-CD20 monoclonal antibody for the treatment of low-grade or transformed low-grade non-Hodgkin's lymphoma (NHL) as an I-131 labeled monoclonal antibody in investigational radioimmunotherapy. The preferred dose is about 450 mg of tositumomab intravenously over about 1 hr. followed by about 35 mg of tositumomab radiolabeled with about 5 mCi of iodine-131 over about 0.5 hr.

As used herein, trastuzumab (Herceptin®) refers to a recombinant DNA-derived humanized monoclonal antibody which targets cancer cells that overexpress the cell-surface protein, HER-2 or erb B2. Trastuzumab slows or stops the growth of these cells. Trastuzumab is used to treat cancers that overexpress the HER-2 protein, for example, the approximately 25 to 30 percent of breast cancers that overexpress HER-2. Trastuzumab is a humanized monoclonal antibody that selectively binds with high affinity in a cell-based assay to the extracellular domain of the human epidermal growth factor receptor 2 protein, HER2. The antibody is an IgG1 kappa that contains human framework regions with the complementarity-determining regions of a murine antibody (4D5) that binds to HER2.

The preferred initial loading dose is between about 3 mg/kg to about 5 mg/kg, more preferably, between about 3.5 mg/kg to about 4.5 mg/kg, and most preferably, about 4 mg/kg of trastuzumab administered as a 90-minute intravenous infusion. The preferred weekly maintenance dose is about 1 mg/kg to about 3 mg/kg, more preferably, between about 1.5 mg/kg to about 2.5 mg/kg, and most preferably, 2 mg/kg trastuzumab. The preferred route of administration is intravenous, more preferably by intravenous infusion over about a 30-minute period. The present invention also encompasses pharmaceutical compositions comprising an effective amount of trastuzumab to be administered in combination with a gallium compound in accordance with the methods of the present invention.

As used herein, antisense molecule refers to a molecule selected from the group including but not limited to an antisense oligomer including oblimersen sodium (G3139, Genasense™). As used herein, antisense oligomer means an antisense oligonucleotide or an analogue or derivative thereof, and refers to a range of chemical species that recognize polynucleotide target sequences through Watson-and-Crick hydrogen bonding interactions with the nucleotide bases of the target sequences. The target sequences may be RNA or DNA, and may be single-stranded or double-stranded. Target molecules include, but are not limited to, pre-mRNA, mRA, and DNA. As used herein, oblimersen sodium refers to a compound which is directed to the mRNA of the bcl-2 gene, a proto-oncogene involved in the inhibition of apoptosis (programmed cell death) of cancerous cells and is believed to be important in a number of solid tumor and hematological malignancies including non-Hodgkin's lymphoma, malignant melanoma, breast, colorectal, ovarian and prostate carcinomas. The protein produced by the bcl-2 gene has two known critical functions in the progression of neoplastic disease: immortalizing cancer cells, creating a survival advantage of malignant over normal cells, and conferring resistance to radiation and chemotherapy, rendering these treatments ineffective in late-stage cancers. High levels of the bcl-2 protein are associated with a poor clinical prognosis for certain cancer patents. G3139 is designed to inactivate the RNA that produces the bcl-2 protein product, thereby preventing cellular production of the protein.

In accordance with the present invention, the dose of oblimersen sodium to be administered in the combination ranges from about 0.01 mg/kg/day to about 50 mg/kg/day; preferably at a dose of about 4 mg/kg/day to about 9 mg/kg/day, and more preferably at a dose of about 5 mg/kg/day to about 7 mg/kg/day. In accordance with the present invention, a time period for administering the bcl-2 antisense is less than 14 days, ranging from about 2 days to about 13 days; preferably ranging from about 3 days to about 9 days, more preferably ranging from about 4 days to about 8 days, or most preferably about 5 days. The present invention also encompasses pharmaceutical compositions comprising an effective amount of one or more bcl-2 antisense oligomers to be administered with a gallium compound in accordance with the methods of the present invention. The pharmaceutical compositions encompass a dose of bcl-2 antisense oligomer ranging from about 0.01 mg/kg/day to about 50 mg/kg/day; preferably at a dose of about 4 mg/kg/day to about 9 mg/kg/day, and more preferably at a dose of about 5 mg/kg/day to about 7 mg/kg/day, in combination with a pharmaceutically acceptable carrier. The pharmaceutical compositions of the present invention are formulated to be delivered as a continuous infusion, or in one or more bolus administrations, or in one or more administrations during a treatment cycle.

In another embodiment, the present invention also encompasses the combination of a gallium compound, oblimersen sodium (G3139) and one or more additional NCAA.

As used herein, anti-telomerase agent refers to molecules which include inhibitors of the enzyme, telomerase, antisense small molecules, or oligomers (e.g., GRN163). GRN163 is a short, modified thiophosphoraamidate oligonucleotide drug that acts as an inhibitor of the enzymatic activity of telomerase. More specifically, it is a telomerase template antagonist. See WO 01/18015. During tumor progression, telomerase is abnormally reactivated and expressed in all major cancer types. The activation of telomerase enables cancer cells to maintain telomere length and resist apoptosis (programmed cell death), enabling unlimited cell growth and resistance to cytotoxic drugs. The present invention also encompasses pharmaceutical compositions comprising an effective amount of one or more anti-telomerase agents to be administered in combination with a gallium compound in accordance with the methods of the present invention. See WO 01/18015, U.S. Pat. Nos. 5,837,835, 5,726,297, 5,824,793, 5,859,233, PCT/US00/24688 which are hereby incorporated by reference in their entirety.

As used herein, aptamer refers to double stranded DNA or single stranded RNA molecules selected from random pools based on their ability to bind specific molecular targets, such as nucleic acid, proteins, small organic compounds, metabolites or organisms.

As used herein, biologic response modifier refers to molecules which include interleukin-2 (IL-2, aldesleukin, Proleukin®), interleukin-11 (IL-11), interleukin-12 (IL-12), or interferon-alpha2a (IFN-α2a). Aldesleukin is a human recombinant IL-2 lymphokine produced by recombinant DNA technology using a genetically engineered E. coli strain containing an analog of the human interleukin-2 gene. Genetic engineering techniques were used to modify the human IL-2 gene, and the resulting recombinant expression clone encodes a modified human interleukin-2 which differs from native interleukin-2 in defined ways. In vitro, recombinant IL-2, aldesleukin, exhibits immunoregulatory properties, including enhancement of lymphocyte mitogenesis and stimulation of long-term growth of human interleukin-2 dependent cell lines, enhancement of lymphocyte cytotoxicity, induction of killer cell (lymphokine-activated (LAK) and natural (NK)) activity, and induction of interferon-gamma production. The in vivo administration of aldesleukin in animals and humans exhibits dose dependent immunological effects, including activation of cellular immunity with profound lymphocytosis, eosinophilia, and thrombocytopenia, and the production of cytokines including tumor necrosis factor, IL-1 and gamma interferon. In vivo experiments in murine tumor models have shown inhibition of tumor growth.

In accordance with the present invention, the dosage of aldesleukin, recombinant human IL-2, would be known to one of skill in the art as suggested by the Physicians' Desk Reference, 56^{th} Ed. (2002) or a similar reference. The preferred course of treatment consists of about two 5-day treatment cycles separated by a rest period. The preferred dose of about 500,000 IU/kg to about 700,000 IU/kg, the more preferred dose of about 550,000 IU/kg to about 650,000 IU/kg, and the most preferred dose is about 600,000 IU/kg (0.037 mg/kg) administered about every 8 hours by intravenous infusion for a maximum of about 14 doses. Following 9 days of rest, the preferred dosage schedule is repeated for another 14 doses, for a maximum of about 28 doses per course, as tolerated. The most preferred aldesleukin dosage treatment regimen is administered by a 15-minute intravenous infusion every 8 hours. The present invention also encompasses pharmaceutical compositions comprising an effective amount of a biologic response modifier, preferably aldesleukin, to be administered in combination with a gallium compound in accordance with the methods of the present invention.

As used herein, bisphosphonate refers to molecules which include alendronate and zoledronic acid (Zometa®). As used herein, zoledronic acid (Zometa®) refers to a bisphosphonic acid in the form of zoledronic acid monohydrate which is an inhibitor of osteoclastic bone resorption. Zoledronic acid monohydrate is designated chemically as (1-hydroxy-2-imidazol-1-yl-phosphonoethyl) phosphonic acid monohydrate. In accordance with the present invention, the dosage of zoledronic acid monohydrate is known to one of skill in the art as suggested by the Physicians' Desk Reference, 56^{th} Ed. (2002) or a similar reference; the preferred dosage is between about 1 mg to about 6 mg, more preferably between about 2 mg to about 5 mg, and most preferably about 4 mg. The preferred route of administration of the dosage is by intravenous infusion. In accordance with the present invention, a time period for administering the dosage of zoledronic acid monohydrate is preferably over no less than 15 minutes duration, more preferably over longer than 15 minutes duration, repeated every three or four weeks for a time interval of between about 9 months and about 15 months, most preferably about 12 months, in accordance with the disease under treatment which would be known to one skilled in the art. The present invention also encompasses pharmaceutical compositions comprising an effective amount of a bisphosphonate compound, e.g., zoledronic acid monohydrate or alendronate to be administered in combination with a gallium compound in accordance with the methods of the present invention.

As used herein, cytoxic fusion protein refers to molecules which include recombinant immunotoxins, e.g., BL22, or denileukin diftitox (Ontak®). As used herein, BL22 refers to a recombinant Pseudomonas exotoxin-based immunotoxin, comprised of the disulfide-stabilized Fv portion of the anti-CD22 antibody, RFB4, genetically fused to a truncated form of Pseudomonas exotoxin A. As used herein, denileukin diftitox (Ontak®) refers to a fusion protein, a recombinant DNA-derived cytotoxic protein produced in an E. coli expression system by genetically fusing protein fragments from the diphtheria toxin to interleukin-2 (IL-2), a naturally occurring immune system protein. This stable, fusion protein targets cells with receptors for IL-2 on their surfaces, including malignant cells and some normal lymphocytes, resulting in cell death. Denileukin diftitox is used in the biologic treatment for persistent or recurrent cutaneous t-cell lymphoma, (CTCL), non-Hodgkin's lymphoma whose malignant cells express the CD25 component of the IL-2receptor (IL-2R). The preferred dosage treatment cycle of denileukin diftitox is between about 8 μg/kg/d to about 10 μg/kg/d or between about 16 μg/kg/d to about 20 μg/kg/d, more preferably about 9 μg/kg/d to about 18 μg/kg/d, for about five consecutive days every 21 days administered intravenously over at least 15 minutes but not longer than about 80 minutes. The preferred maximum number of treatment cycles is between about 1 cycle and about 8 cycles, more preferably between about 2 cycles and about 6 cycles, and most preferably about 4 cycles. The present invention also encompasses pharmaceutical compositions comprising an effective amount of a cytoxic fusion protein, preferably denileukin diftitox, to be administered in combination with a gallium compound in accordance with the methods of the present invention.

As used herein, immunomodulatory agent refers to molecules selected from the group including but not limited to thalidomide (Thalomid®). As used herein, thalidomide refers to α-(N-phthalimido)glutarimide, an immunomodulatory agent whose effects are variable but may be related to suppression of excessive tumor necrosis factor-alpha (TNF-α) production and down-modulation of selected cell surface adhesion molecules involved in leukocyte migration. Thalidomide has efficacy in the treatment of hematologic malignancies, including lymphomas and plasma cell disorders, e.g., myeloma. The preferred initial dosage of thalidomide is about 50 mg/day to about 300 mg/day, administered once daily, up to about 800 mg/day, more preferably up to about 200 mg/day to about 400 mg/day. Preferably, thalidomide is administered daily for a period to be readily determined by one of ordinary skill in the art. The present invention also encompasses pharmaceutical compositions comprising an effective amount of an immunomodulatory agent, preferably thalidomide, to be administered in combination with a gallium compound in accordance with the methods of the present invention.

As used herein, anti-angiogenesis agent refers to molecules that limit or inhibit the development and/or proliferation of blood vessels, e.g., VEGF receptor agonists including antagonists, such as, VEGF inhibitors, thalidomide endostatin modulators and the like. One embodiment of the present invention comprises the administration of a gallium compound, preferably gallium nitrate, in combination with an anti-angiogenesis agent.

As used herein, immunostimulatory agent refers to molecules which include CpG oligodeoxynucleotides (CpG 7909) and bis-CpG oligodeoxynucleotides. The present invention also encompasses pharmaceutical compositions comprising an effective amount of an immunostimulatory agent to be administered in combination with a gallium compound in accordance with the methods of the present invention.

As used herein, molecular decoy refers to molecules which include decoy tumor necrosis factor receptors (TNFR), e.g., DcR1 and DcR2 which are membrane associated decoy molecules which compete with the receptors, DR4 and DR5, for their binding to their respective ligands, and DcR3, a soluble decoy receptor, which inhibits ligand-induced apoptosis or programmed cell death. Molecular decoys may promote or inhibit signal transduction by acting as a decoy to bind ligand and therefore block ligand binding to its native receptor which interfere with transduction of the signal to the nucleus of the cell.

As used herein, molecular inhibitor refers to molecules which include P-glycoprotein inhibitor (valdospar, PSC-833). As used herein, P-glycoprotein inhibitor refers to a compound which modulates the multidrug transporter, P-glycoprotein (Pgp), which is a cellular efflux pump. The P-glycoprotein inhibitor, valdospar (PSC 833), is a cyclosporin D analogue which causes apoptosis of cancer cells and induces a rise in the intracellular levels of ceramide. Intrinsic and acquired multidrug resistance (MDR) in many human cancers may be due to expression of the Pgp, which is encoded by the mdr1 gene. There is substantial evidence that Pgp is expressed both as an acquired mechanism (e.g., in leukemias, lymphomas, myeloma, and breast and ovarian carcinomas) and constitutively (e.g., in colorectal and renal cancers) and that its expression is of prognostic significance in many types of cancer. MDR modulation may delay the emergence of clinical drug resistance and prevent drug resistance in the earlier stages of disease. PSC 833 significantly increases the paclitaxel and doxorubicin exposure secondary to decreased clearance, accounting for the need to reduce doses 2-fold to achieve equivalent myelosuppression. (Advani et al., Clinical Cancer Research 7(5) 1221-9 (2001)). The preferred dosage of PSC 833 is 5 mg/kg p.o. four to six times daily for a preferred total of 12 doses with chemotherapy preferably administered on day 2, preferably after the fifth or sixth dose of PSC 833. Cycles of administration preferably are between about once weekly to about every 3 to 4 weeks. The present invention also encompasses pharmaceutical compositions comprising an effective amount of a molecular inhibitor, preferably P-glycoprotein inhibitor, more preferably PSC 833, to be administered in combination with a gallium compound in accordance with the methods of the present invention.

As used herein, proteasome inhibitor refers to molecules selected from the group including but not limited to bortezomib (Velcade®). The proteasome is an enzyme complex found within the cytoplasm of cells. Evidence suggests that it serves both as a disposal system for damaged cellular proteins and as a mechanism for degrading short-lived regulatory proteins that govern cellular functions such as the cell cycle, cell growth, and differentiation. Because these processes or their disregulation are crucial steps in tumor formation, the proteasome pathway is a logical target for therapeutic intervention. Selective inhibition of proteasome activity has numerous effects that can be relevant in cancer treatment, including attenuating the activity of NF-kB, the transcription factor that controls cellular inflammatory response, and inhibiting the activity of bcl-2, a gene involved in cell survival. Elevated NF-kB and bcl-2 activities allow cancer cells to defend themselves against treatment with standard chemotherapy agents. By blocking the normal function of NF-kB and bcl-2, a proteasome inhibitor can cause the death of cancer cells. The preferred dose of bortezomib is between about 1.0 mg/m² and 1.3 mg/m², with the most preferred dose at about 1.3 mg/m², administered by intravenous push on about days 1, 4, 8, and 11 of a 21-day cycle for up to about eight cycles. The present invention also encompasses pharmaceutical compositions comprising an effective amount of a proteasome inhibitor, preferably bortezomib, to be administered in combination with a gallium compound in accordance with the methods of the present invention.

As used herein, protein kinase inhibitor refers to molecules which include a protein tyrosine kinase inhibitor, e.g., imatinib mesylate (Gleevec®) and gefitinib (Iressa®), or a protein kinase C inhibitor, e.g., ruboxistaurin mesylate (LY333531®). As used herein, imatinib mesylate (Gleevec®) refers to a protein-tyrosine kinase inhibitor that inhibits the Bcr-Abl tyrosine kinase, the constitutive abnormal tyrosine kinase created by the Philadelphia chromosome abnormality in chronic myeloid leukemia (CML). Imatinib mesylate is designated chemically as 4-[(4-methyl-1-piperazinyl)methyl]-N-[4-methyl-3-[[4-(3-pyridinyl)-2-pyrimidinyl]-amino]phenyl]benzamide methanesulfonate. Imatinib mesylate also inhibits the receptor tyrosine kinases for platelet-derived growth factor (PDGF) and stem cell factor (SCF), c-kit, and inhibits PDGF- and SCF-mediated cellular events. In, vitro, imatinib mesylate inhibits proliferation and induces apoptosis in gastrointestinal stromal tumor (GIST) cells, which express an activating c-kit mutation. The preferred dosage of imatinib mesylate is from about 300 mg/day to about 500 mg/day with the most preferred dosage at about 400 mg/day, administered orally, once daily, for patients in chronic phase CML, and from about 500 mg/day to about 700 mg/day with the most preferred dosage at about 600 mg/day, administered orally, once daily, for patients in accelerated phase or blast crisis, for as long as the patient continues to benefit. More preferred dose increases as tolerated are from about 400 mg to about 600 mg in patients with chronic phase disease, or from about 600 mg to about 800 mg (given as 400 mg twice daily) in patients in accelerated phase or blast crisis. As used herein, gefitinib (Iressa®) refers to an anilinoquinazoline with the chemical name 4-Quinazolinamine, N-(3-chloro-4-fluorophenyl)-7-methoxy-6-[3-4-morpholin) propoxy] with a molecular formula C₂₂H₂₄ClFN₄O₃. Gefitinib inhibits the intracellular phosphorylation of numerous tyrosine kinases associated with transmembrane cell surface receptors, including the tyrosine kinases associated with the epidermal growth factor receptor (EGFR-TK). EGFR is expressed on the cell surface of many normal cells and cancer cells. The dosage of gefitinib is known to one of skill in the art as suggested by the Physicians' Desk Reference, 56^{th} Ed. (2002) or a similar reference; the preferred dose is about 250 mg/d.

As used herein, ruboxistaurin mesylate (LY333531) refers to a protein kinase C β (PKC β) inhibitor, which delays the progression of diabetic retinopathy and diabetic macular edema and improves diabetic peripheral neuropathy. The preferred dose of ruboxistaurin mesylate is between about 32 mg to about 64 mg, with the most preferred dose being about 32 mg. The present invention also encompasses pharmaceutical compositions comprising an effective amount of a protein kinase inhibitor, preferably imatinib mesylate or ruboxistaurin mesylate to be administered in combination with a gallium compound in accordance with the methods of the present invention.

As used herein, retinoid refers to a molecule that selectively binds and/or activates retinoic acid receptors or retinoid receptors and include but are not limited to bexarotene (Targretin®) or tretinoin (Vesanoid®). As used herein, bexarotene (Targretin®) refers to a retinoid that selectively binds and activates retinoid X receptor subtypes (RXRα, RXRβ, RXRγ). RXRs can form heterodimers with various receptor partners such as retinoic acid receptors (RARs), vitamin D receptor, thyroid receptor, and peroxisome proliferator activator receptors (PPARs). Once activated, these receptors function as transcription factors that regulate the expression of genes that control cellular differentiation and proliferation. Bexarotene inhibits the growth in vitro of some tumor cell lines of hematopoietic and squamous cell origin. It also induces tumor regression in vivo in some animal models. The exact mechanism of action of bexarotene in the treatment of cutaneous T-cell lymphoma (CTCL) is unknown.

Bexarotene is supplied in oral dosage form as capsules or as a 1% gel for topical application to CTCL lesions. The preferred initial dose of bexarotene capsules is between about 100 mg/m²/d and about 1,000 mg/m²/d, with the preferred dose between about 300 mg/m²/d to about 400 mg/m²/d, with the most preferred dose at about 300 mg/m²/d administered indefinitely while benefit accrues. The preferred initial dose of bexarotene 1% gel is between about once every other day to about 4 times daily, with the most preferred frequency of administration at about 4 times daily.

As used herein, tretinoin (Vesanoid®) refers to a retinoid that induces maturation of acute promyelocytic leukemia (APL) cells in culture. Chemically, tretinoin is all-trans retinoic acid and is related to retinol (Vitamin A). The preferred dose of tretinoin for induction of remission in APL is from about 40 mg/m²/d to about 50 mg/m²/d, most preferably about 45 mg/m²/d, administered as two evenly divided doses until complete remission is documented. Preferably, therapy should be discontinued 30 days after achievement of complete remission or after 90 days of treatment, whichever occurs first. The present invention also encompasses pharmaceutical compositions comprising an effective amount of a retinoid, preferably bexarotene or tretinoin, to be administered in combination with a gallium compound in accordance with the methods of the present invention.

As used herein, transcription factor refers to molecules which include nuclear factor-kappa B (NF-κB). As used herein, nuclear factor-kappa B (NF-kB) refers to a cellular protein involved in cell signaling pathways that regulate the transcription of key genes involved in several diseases, including but not limited to inflammation (e.g., atherosclerosis, arthritis, inflammatory bowel disease, rheumatoid arthritis and septic shock), malignant transformation and tumor proliferation (e.g., certain blood cancers and solid tumors), and bone rebuilding (e.g., osteoporosis). The present invention also encompasses pharmaceutical compositions comprising an effective amount of a transcription factor, preferably NF-κB, to be administered in combination with a gallium compound in accordance with the methods of the present invention.

As used herein, arsenic compound refers to arsenic-containing molecules which include arsenic trioxide (Trisenox®). Arsenic trioxide refers to an agent which induces remission and consolidation in patients with acute promyelocytic leukemia (APL) who are refractory to, or who have relapsed from, retinoid and anthracycline-based chemotherapy, and whose APL is characterized by the presence of the t(15;17) translocation or PML/RAR-alpha gene expression. Arsenic trioxide appears to have multiple targets and mechanisms of antileukemic activity: it degrades a protein that causes abnormal levels of immature white blood cells while directly inducing apoptosis. Arsenic trioxide has orphan drug designation from the FDA for APL, multiple myeloma, myelodysplastic syndromes (MDS), chronic myeloid leukemia (CML), and acute myeloid leukemia (AML). For induction of remission, arsenic trioxide is administered intravenously at a preferred dose of about 0.15 mg/kg daily until bone marrow remission is achieved. Total induction dose preferably should not exceed about 60 doses. For consolidation, treatment should begin about 3 to about 6 weeks after completion of induction therapy. Arsenic trioxide is administered intravenously at a preferred dose of about 0.15 mg/kg daily for about 25 doses over a period up to about 5 weeks. The present invention also encompasses pharmaceutical compositions comprising an effective amount of arsenic compounds, preferably arsenioxide trioxide, to be administered in combination with a gallium compound in accordance with the methods of the present invention.

Routes of administration of the NCAA of the present invention include but are not limited to administration systemically, regionally or locally, by enteral or parenteral means, wherein the routes of administration include but are not limited to intravenously, intra-arterially, intraperitoneally, intrathecally, orally, sublingually, rectally, intracutaneously, subcutaneously, percutaneously, transcutaneously, intradermally or intramuscularly. Exemplary dose ranges used for particular therapeutic agents employed for specific diseases can be found in the Physicians' Desk Reference, 56^{th} Edition (2002).

Administration of the gallium compound and the NCAA of the present invention may be administered simultaneously, either in the same or different pharmaceutical formulation, or sequentially. If administered sequentially, the delay in administering the second (or additional) active ingredient should not be such as to lose the benefit of the efficacious effect of the combination of the active ingredients. It is understood that the duration and frequency of administration of the gallium compound and the NCAA are determined by the nature of the particular compound, therefore, the NCAA may be administered more frequently, less frequently, or at the same frequency as the gallium compound. The gallium compound and the NCAA are administered intermittently or continuously. More preferably the gallium compound is administered from about 2 to about 12 hours before the NCAA.

Pharmaceutical compositions comprising the NCAA of the present invention are encompassed by the present invention. Specific methods for preparing administrable compositions will be known or apparent to those skilled in the art and are described in more detail in, for example, Remington's Pharmaceutical Science, 15^{th} Ed., Mack Publishing Company, Easton, Pa. (1980), which is incorporated herein by reference.

As used herein, pharmaceutically acceptable carrier refers to a carrier medium that does not interfere with the effectiveness of the biological activity of the active ingredient. The carrier medium is essentially chemically inert and nontoxic. As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, carrier refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic lipids, including, but not limited to N-(1(2,3-dioleyloxy)propyl)-N,N,N-trimethylammonium chloride (DOTMA) and diolesylphosphotidylethanolamine (DOPE). Liposomes are also suitable carriers for the antisense oligomers of the invention. Such compositions should contain a therapeutically effective amount of the compound, together with a suitable amount of carrier so as to provide the form for proper administration to the patient. The formulation should suit the mode of administration.

As used herein, pharmaceutically acceptable salts refers to salts prepared from pharmaceutically acceptable, essentially nontoxic, acids and bases, including inorganic and organic acids and bases. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

All of the texts cited above are hereby incorporated herein by reference.

What is claims is:

1. A treatment regimen for a mammal with neoplastic disease, comprising the steps of administering a therapeutic dose of a gallium compound and administering a therapeutic dose of at least one nonchemotherapeutic anticancer agent (NCAA).

2. The method of claim 1 wherein the gallium compound and NCAA are administered simultaneously.

3. The method of claim 1 wherein administration of the gallium compound and NCAA are separated by a selected time interval.

4. The method of claim 1 wherein the gallium compound is gallium nitrate.

5. The method of claim 1 wherein the NCAA is at least one compound selected from the group consisting of an antibody, a small molecule, an antisense molecule, an anti-telomerase agent, a biologic response modifier, a bisphosphonate, a cytotoxic fusion protein, an immunomodulatory agent, an immunostimulatory agent, a molecular inhibitor, a proteasome inhibitor, a protein kinase inhibitor, a retinoid, a transcription factor and an arsenic compound.

6. The method of claim 5 wherein the NCAA is at least one antibody, selected from the group consisting of a monoclonal antibody, a genetically engineered antibody, a bispecific antibody, a humanized antibody, a chimeric antibody, an antibody fragment, a single-chain antibody, an scFv fragment, an Fab fragment, an F(ab)′ fragment, and an (Fab)′₂ fragment.

7. The method of claim 6 wherein the antibody is selected from the group consisting of alemtuzumab, cetuximab, epratuzumab (LL2, hLL2), gemtuzumab ozogamicin, ibritumomab tiuxetan, rituximab, tositumomab, trastazumab, and anti-CD19/anti-CD3 single-chain bispecific antibody (bscCD19xCD3).

8. The method of claim 5 wherein the NCAA is the antisense molecule oblimersen sodium.

9. The method of claim 5 wherein the NCAA is an anti-telomerase agent selected from the group consisting of an antisense molecule, a small molecule and an oligomer.

10. The method of claim 9 wherein the anti-telomerase agent is GRN163.

11. The method of claim 1 wherein the NCAA is an aptamer.

12. The method of claim 5 wherein the NCAA is a biologic response modifier selected from the group consisting of interleukin-2 (IL-2, aldesleukin), interleukin-11 (IL-11), interleukin-12 (IL-12), and interferon-alpha2a (IFN-α2a).

13. The method of claim 5 wherein the NCAA is a bisphosphonate.

14. The method of claim 5 wherein the NCAA is the cytotoxic fusion protein denileukin difititox.

15. The method of claim 5 wherein the NCAA is the immunomodulatory agent thalidomide.

16. The method of claim 5 wherein the NCAA is the immunostimulatory agent CpG oligodeoxynucleotide.

17. The method of claim 1 wherein the NCAA is a molecular decoy.

18. The method of claim 5 wherein the NCAA is the molecular inhibitor P-glycoprotein inhibitor.

19. The method of claim 5 wherein the NCAA is the proteasome inhibitor bortezomib.

20. The method of claim 5 wherein the NCAA is a protein kinase inhibitor selected from the group consisting of a protein tyrosine kinase inhibitor and a protein kinase C inhibitor.

21. The method of claim 20 wherein the protein tyrosine kinase inhibitor is imatinib mesylate or the protein kinase C inhibitor is ruboxistaurin mesylate.

22. The method of claim 5 wherein the NCAA is a retinoid selected from the group consisting of bexarotene and tretinoin.

23. The method of claim 5 wherein the NCAA is the transcription factor nuclear factor-kappa B (NF-κB).

24. The method of claim 5 wherein the NCAA is arsenic trioxide.

25. The method of claim 1 wherein the NCAA is a compound directed to a target molecule selected from the group consisting of CD52 antigen, epidermal growth factor receptor, CD22 receptor, CD33 antigen, CD20 antigen, HER-2 receptor, CD19 antigen and CD3 antigen.
